(19) United States
(12) Patent Application Publication
(10) Pub. No.: US 2025/0143665 A1
(43) Pub. Date: May 8, 2025

(54) TUBULAR MEMBER FOR FACILITATING THE COLLECTION OF SOUND WAVES ORIGINATING INSIDE A LIVING BODY

(71) Applicant: Heroic Faith Medical Science Co., Ltd., New Taipei City (TW)

(72) Inventors: Yuan Ren Cheng, Taipei City (CN); Fushun Hsu, Taipei City (CN)

(21) Appl. No.: 19/018,608

(22) Filed: Jan. 13, 2025

Related U.S. Application Data
(63) Continuation of application No. PCT/US2023/027656, filed on Jul. 13, 2023.
(60) Provisional application No. 63/368,275, filed on Jul. 13, 2022.

(51) Int. Cl. A61B 7/04

(52) U.S. Cl. CPC A61B 7/04; A61B 2562/0204; A61B 2562/166

(57) ABSTRACT

Introduced here is an acoustic collection device that is designed to extend the path along which sound waves can be collected by a microphone. An acoustic collection device can include a tubular body that has (i) a distal interface through which sound waves corresponding to sounds internal to a living body are collected and (ii) a proximal interface through which the sound waves are presented to a microphone. A channel defined by the inner surface of the tubular body extends between the distal and proximal interfaces. The channel allows the sound waves to travel from the distal interface to the proximal interface. The acoustic collection device could be connected to the housing of an electronic device that is used to record the sound waves, or the acoustic collection device could be implemented in an input unit for an electronic stethoscope system.

### CROSS-REFERENCE TO RELATED APPLICATIONS

This application is a continuation of International Application No. PCT/US2023/027656, filed on Jul. 13, 2023, which claims priority to U.S. Provisional Application No. 63/368,275, filed on Jul. 13, 2022, each of which is incorporated herein by reference in its entirety.

### TECHNICAL FIELD

Various embodiments pertain to tubular members designed to facilitate the recording of sound waves originating inside a living body.

### BACKGROUND

Historically, acoustic stethoscopes have been used to listen to the internal sounds originating inside a living body. This process—referred to as “auscultation”—is often performed for the purpose of examining biological systems whose performance can be inferred from these internal sounds. Normally, acoustic stethoscopes include a single chestpiece that has a rigid (e.g., metallic) resonator designed to be placed against the body and a pair of hollow tubes that are connected to earpieces. As sound waves are captured by the resonator, they are directed to the earpieces via the pair of hollow tubes.

But acoustic stethoscopes suffer from several drawbacks. For example, an acoustic stethoscope will attenuate the sound proportional to the frequency of the source. Thus, the sound conveyed to the earpieces tends to be very faint, which can make it difficult to accurately diagnose conditions. In fact, due to variation in sensitivity of the ear, some sounds (e.g., those below 50 hertz) may not be heard at all.

Some enterprises have begun developing electronic stethoscopes (also called “digital stethoscopes” or “stethophones”) to address the drawbacks of acoustic stethoscopes. Electronic stethoscopes improve upon acoustic stethoscopes by electronically amplifying sounds. For instance, an electronic stethoscope may address the faint sounds originating inside a living body by amplifying these sounds. To accomplish this, the electronic stethoscope converts sound waves detected by a microphone located in the chestpiece into an electrical signal and then amplifies the electrical signal for optimal listening.

### BRIEF DESCRIPTION OF THE DRAWINGS

FIG. 1 is a perspective view of an acoustic collector that is designed to extend the path along which sound waves can be collected.

FIG. 2 is a perspective view of another acoustic collector that is designed to extend the path along which sound waves can be collected.

FIG. 3 is a side cross-sectional view of an acoustic collector in a non-deformed state.

FIG. 4 illustrates an example of an acoustic collector in which the distal and proximal portions of the tubular body include depressions.

FIG. 5 includes several examples of different channel geometries.

FIG. 6 is a side view of an acoustic collector.

FIG. 7 includes side and overhead views illustrating how an acoustic collector can be secured to an electronic device and then placed against the surface of a body.

FIG. 8 depicts a flow diagram of a process for manufacturing an acoustic collector.

FIG. 9 depicts a flow diagram of a process for acquiring audio data indicative of sounds internal to a living body.

FIG. 10A includes a top perspective view of an input unit for an electronic stethoscope system.

FIGS. 10B-C include bottom perspective views of the input unit of FIG. 10A.

FIG. 11A includes a cross-sectional side view of an input unit for an electronic stethoscope system that does not include an acoustic collector.

FIG. 11B includes a cross-sectional perspective view of the input unit of FIG. 11A.

FIG. 11C includes a cross-sectional side view of the input unit of FIG. 11A.

FIG. 12A includes a cross-sectional perspective view of an input unit that does include an acoustic collector.

FIG. 12B includes a cross-sectional side view of the input unit of FIG. 12A.

FIG. 13 illustrates how one or more input units can be connected to a hub unit to form an electronic stethoscope system.

FIG. 14 is a high-level block diagram illustrating exemplary components of an input unit and a hub unit of an electronic stethoscope system.

Embodiments are illustrated by way of example and not limitation in the drawings. While the drawings depict various embodiments for the purpose of illustration, those skilled in the art will recognize that alternative embodiments may be employed without departing from the principles of the technology. Accordingly, while specific embodiments are shown in the drawings, the technology is amenable to various modifications.

### DETAILED DESCRIPTION

Electronic stethoscopes show significant promise as the impact of sounds originating outside of the living body under examination, commonly called “ambient noise,” can be mitigated through electronic identification, filtration, amplification, and physical isolation. But electronic and acoustic stethoscopes both suffer from a notable downside, namely, that these are sophisticated medical devices that cannot be used by individuals who have not been properly trained. For this reason, electronic and acoustic stethoscopes are generally not suitable for use outside of healthcare facilities, except by trained healthcare professionals. This is problematic as many patients have begun enrolling in telemedicine programs in which healthcare services are provided remotely.

Introduced here is an acoustic collection device (also referred to as an “acoustic concentration device”) that is designed to extend the path along which sound waves can be collected by a microphone housed in an electronic device. For convenience, the acoustic collection device may be referred to as an “acoustic collector” or “acoustic concentrator.” An acoustic collector can include a tubular body—generally comprising a deformable material—that has (i) a distal interface through which sound waves corresponding to sounds internal to a living body are collected and (ii) a proximal interface through which the sound waves are presented to a microphone of an electronic device to which the tubular body is connected. A channel defined by the inner surface of the tubular body extends between the distal and proximal interfaces. The channel allows the sound waves to travel from the distal interface to the proximal interface.

At a high level, the acoustic collector can act as a guide by directing sounds that originate from a space adjacent to the electronic device toward the microphone. Assume, for example, that an individual is interested in collecting sound waves that are representative of sounds internal to a living body (or simply “body”). In such a scenario, the individual may affix an acoustic collector to an electronic device and then position the electronic device such that the acoustic collector is able to collect sound waves originating in the living body and then direct those sound waves toward the microphone. Normally, this is accomplished by holding the acoustic collector against the body, either directly (e.g., against the skin) or indirectly (e.g., against clothes). Such an approach allows internal sounds to be recorded by the microphone while external sounds are muted or filtered. The term “internal sounds” refers to those acoustic sounds that originate inside the body, while the term “external sounds” refers to those acoustic sounds that originate outside the body.

In some embodiments, the acoustic collector comprises a porous material such as an open cell foam or a closed cell foam. Open cell foams are commonly made of polyurethane, polyvinyl chloride (“PVC”), nitrile, silicone, or ethylene propylene diene monomer (“EPDM”) rubber, while closed cell foams are commonly made of ethylene-vinyl acetate (“EVA”), polyethylene, neoprene, PVC, nitrile, or styrene-butadiene rubber. There are several benefits to using foams. First, foams generally provide high resistance to compression set (i.e., collapse from pressure, as well as high resiliency, vibration damping, and impact absorption. Second, foams generally perform well in absorbing errant sound waves. Consider, for example, a scenario in which an acoustic collector having a tubular body with a cylindrical cavity defined therethrough is pressed against the surface of a living body. Sound waves that travel along the length of the cylindrical cavity parallel to its wall will reach the microphone of the electronic device without issue. However, errant sound waves-namely, those sound waves that do not travel parallel to the wall of the cylindrical cavity-will strike the wall of the cylindrical cavity. If the tubular body comprises foam, these errant sound waves may be largely, if not entirely, absorbed-leading to “cleaner” recording by the microphone of the electronic device.

The acoustic collectors described herein are designed such that they can be used by individuals who are not trained to use sophisticated medical devices like electronic and acoustic stethoscopes. For example, a patient could use an acoustic collector to record her own internal sounds. As another example, a friend or family member could use an acoustic collector to record the internal sounds of a patient. As further discussed below, acoustic collectors can be used in combination with electronic devices that are readily available and, as such, may prove to be especially beneficial as healthcare professionals seek to provide remote diagnoses through telemedicine.

For the purpose of illustration, embodiments may be described in the context of recording internal sounds for the purpose of diagnosing respiratory ailments. However, those skilled in the art will recognize that acoustic collectors could be used to facilitate recording of internal sounds originating from elsewhere in the body. Thus, an acoustic collector could be used to collect sound waves generated by the circulatory system, respiratory system, or gastrointestinal system.

### Terminology

Brief definitions of terms, abbreviations, and phrases used throughout the present disclosure are given below.

The terms “connected,” “coupled,” and variants thereof are intended to include any connection or coupling between two or more elements, either direct or indirect. For example, a pair of objects may be directly connected to one another, or a pair of objects may be indirectly connected to one another via one or more intermediary objects.

The term “about” means within ±10 percent of the recited value.

### Overview of Acoustic Collector

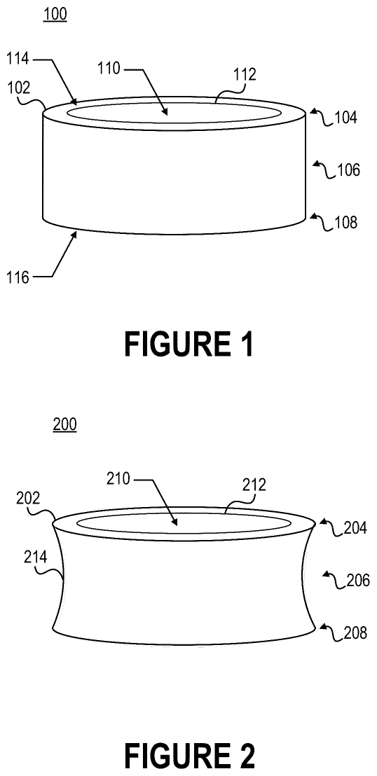

FIG. 1 is a perspective view of an acoustic collector 100 that is designed to extend the path along which sound waves can be collected. As further discussed below, the acoustic collector 100 can serve as a passive mechanism for collecting and then presenting sound waves to a microphone housed in an electronic device. By directing the sound waves toward the microphone, the acoustic collector 100 can significantly improve the ability to record sounds of interest.

In the embodiment shown in FIG. 1, the acoustic collector 100 includes a tubular body 102 having a distal portion 104, medial portion 106, and proximal portion 108. The distal portion 104, medial portion 106, and proximal portion 108 may also be referred to as the “first portion,” “second portion,” and “third portion,” respectively. A channel 110 defined by the inner surface 112 of the tubular body 102 can extend between the distal and proximal portions 104, 108.

As further discussed below, the acoustic collector 100 can be secured to an electronic device. Then, during a recording operation, the distal portion 104 of the acoustic collector 100 can be placed against a living body (or simply “body”) such that sound waves originating in the body are guided toward the microphone of the electronic device. Accordingly, the distal interface 114 located at the distal portion 104 can serve as an ingress point through which sound waves enter the acoustic collector 100. Meanwhile, the proximal interface 116 located at the proximal portion 108 can server as an egress point through which sound waves exist the acoustic collector 100. The distal and proximal interfaces 114, 116 may correspond to the opposing ends of the channel 110.

Normally, at least a portion of the tubular body 102 is comprised of a deformable material capable of deforming from its original form under pressure and then regaining its original form upon removal of the pressure. Examples of deformable materials include elastomeric materials, sponge materials, foam materials, and the like. Because deformable materials are capable of deforming under pressure, tubular bodies comprised of such materials can readily expand and compress along the lateral and longitudinal axes to accommodate various pressures as further discussed below. In some embodiments, the entire tubular body 102 is comprised of a deformable material. In other embodiments, only a portion of the tubular body 102 is comprised of a deformable material while other portion(s) of the tubular body are comprised of a rigid material. For example, the distal portion 104 and medial portion 106 may be comprised of a deformable material while the proximal portion 108 may be comprised of a non-deformable material or less deformable material, so as to provide stability and structure near where the acoustic collector 100 contacts the electronic device.

The tubular body 102 may be designed such that the medial portion 106 deforms when a force is applied generally along a longitudinal axis of the acoustic collector 100. Such force may be applied, for example, if an individual moves the electronic device to which the acoustic collector 100 is connected along the longitudinal axis when the distal portion 104 of the tubular body 102 is held against the surface of a body. Upon application of pressure to the proximal portion 106, the medial portion 106 may partially collapse or otherwise deform toward the distal portion 102. However, the channel 110 may be designed such that sound waves collected through the distal interface 114 are still able to travel through the tubular body 102 toward the proximal interface 116.

FIG. 2 is a perspective view of another acoustic collector 200 that is designed to extend the path along which sound waves can be collected. The acoustic collector 200 shown in FIG. 2 includes several features at least generally similar to the acoustic collector 100 described with reference to FIG. 1. For example, the acoustic collector 200 includes a tubular body 202 that has a distal portion 204, a proximal portion 208 opposite the distal portion 204, and a medial portion 206 spacing the distal and proximal portions 204, 208 apart from each other. A channel 210 defined by the inner surface 212 of the tubular body 202 extends from the distal portion 204 to the proximal portion 208.

As shown in FIG. 2, the outer surface 214 of the tubular body 202 may be non-linear in nature. For example, the tubular body 202 may have a concave sidewall that is arranged adjacent to an outwardly flared portion along the distal end and/or proximal end. Alternatively, the outer surface 214 of the tubular body 202 may be straight as shown in FIG. 1. In some embodiments, the outer surface of the tubular body is tapered, angled, or curved. For example, the outer surface of the tubular body may be curved in a convex manner. As another example, the tubular body may have a ribbed outer surface, with a series of annular structures (also called “ribs”) that are spaced apart, to more easily accommodate compression when force is applied along the longitudinal axis.

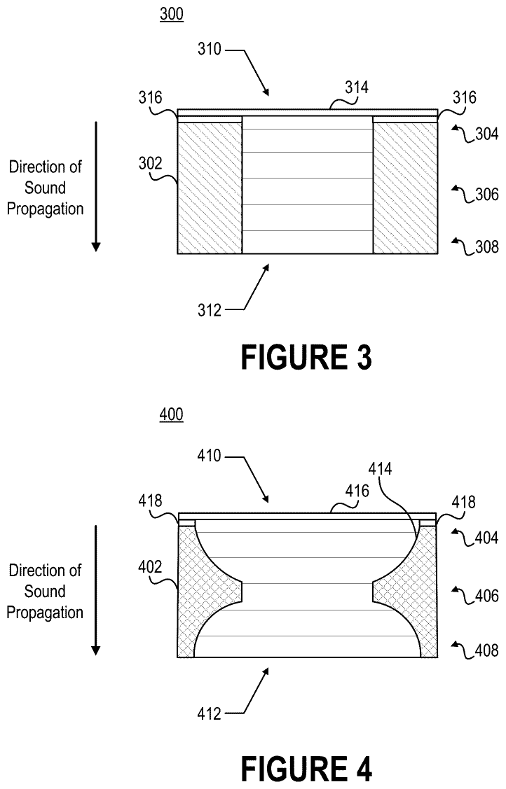

FIG. 3 is a side cross-sectional view of an acoustic collector 300 in a non-deformed state. While the acoustic collector 300 shown in FIG. 3 has a form similar to the acoustic collector 100 described with reference to FIG. 1, those skilled in the art will recognize that the features may be similarly applicable to acoustic collectors having other forms, such as the acoustic collector 200 described with reference to FIG. 2.

The acoustic collector 300 includes a tubular body 302 having a distal portion 304 configured to be positioned proximate to the surface of a body, a proximal portion 308 configured to be positioned proximate to the microphone of an electronic device, and a medial portion 306 that spaces the distal and proximal portions 304, 308 apart from each other. The tubular body 302 may be designed such that the medial portion 306 deforms when a force is applied generally along a longitudinal axis of the acoustic collector 300. Deformation along the longitudinal axis can actually stabilize the tubular body 302, providing rigidity and strengthening the isolation of the sound waves traveling through the channel, thereby resulting in improved auscultation.

As shown in FIG. 3, the acoustic collector 300 can include (i) a first opening 310 at the distal portion 304 at which to collect sound waves and (ii) a second opening 312 at the proximal portion 308 at which to present the sound waves, for example, to a microphone of an electronic device to which the tubular body 302 is secured. Together, the first and second openings 310, 312 may represent the opposing ends of a channel defined through the tubular body 302. Accordingly, as sound waves enter the first opening 310, those sound waves will travel to the second opening 312 though the channel.

In some embodiments, a diaphragm 314 (also called a “vibration film”) extends across the first opening 310 of the channel defined through the tubular body 302. The diaphragm 314 can be used to listen to high-pitch sounds, such as those often produced by the lungs. The diaphragm 314 can be formed from a variety of materials so long the diaphragm 312 is rigid. For example, the diaphragm 312 may be a thin plastic disk comprised of an epoxy-fiberglass compound or glass fibers. As shown in FIG. 3, the diaphragm 314 may extend across the entire diameter of the distal interface of the tubular body 302. That need not necessarily be the case, however. In some embodiments, the diaphragm 314 does not extend across the entire diameter of the distal interface of the tubular body 302. In such embodiments, a portion of the distal interface of the tubular body 302 may be exposed. The diaphragm 314 can serve as the surface vibration collector and need not seal the whole channel or provide an airtight seal of the channel.

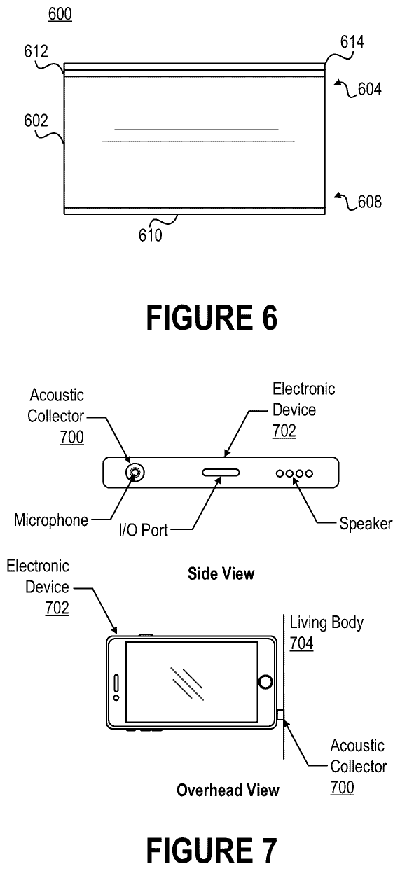

In embodiments where a diaphragm 314 is affixed to the distal interface of the tubular body 302, an adhesive film 316 (also called an “adhesive layer” or simply “adhesive”) may be located along at least part of the distal interface. For example, the adhesive 316 may be in the form of an annular ring that extends around the entire periphery of the distal interface of the tubular body 302. As another example, several “patches” of adhesive 316 may be arranged about the periphery of the distal interface of the tubular body 302. Normally, the adhesive 316 comprises a permanent adhesive so as to prevent the diaphragm 314 from disconnecting from the distal interface during use. When the diaphragm 314 is pulled away from the skin, the diaphragm 314 may experience a slight vacuum force due to, for example, negative fluid pressure of sweat to adhere to the skin. The adhesive 316 should be strong enough to ensure that the entire acoustic collector 300 can be detached from the body without issue. Examples of suitable adhesives include pressure-sensitive adhesives, sealants, and other reactive and non-reactive adhesives. Accordingly, in some embodiments, the adhesive 316 may need to be “activated” through the application of pressure, heat, or light during a manufacturing process. As further discussed below with reference to FIG. 6, an adhesive could also be secured, deposited, or otherwise placed on the distal portion 304 (e.g., along the surface of the diaphragm 314) and/or proximal portion 308 to facilitate securement to the body and electronic device, respectively.

Normally, the diameter of the distal and proximal interfaces is 5-10 millimeters (“mm”). The diameter of the distal interface need not necessarily be the same as the diameter of the proximal interface, however. For example, the distal and proximal interfaces could have diameters of about 6 mm, or the distal interface could have a diameter of about 10 mm while the proximal interface could have a diameter of about 6 mm. The diameter of the first and second openings 310, 312 along the distal and proximal interfaces may be 2-7 mm. For example, in embodiments where the diameter of the distal and proximal interfaces is about 6 mm, the diameter of the first and second openings 310, 312 may be about 3 mm. Depending on the design of the channel, the diameter of the first opening 310 may not be the same as the diameter of the second opening 312. For example, the first opening 310 could have a diameter of about 7 mm if the distal interface has a diameter of about 10 mm, and the second opening 312 could have a diameter of 3 mm if the proximal interface has a diameter of about 6 mm. Normally, the acoustic collector 300 is designed such that the tubular body 302 still has a thickness of at least 1 mm after the channel is formed.

In some embodiments, the first opening and/or the second opening are defined by, or extend from, depressions in the distal portion and/or proximal portion, respectively. As an example, FIG. 4 illustrates an example of an acoustic collector 400 in which the distal and proximal portions 404, 408 of the tubular body 402 include depressions. The distal portion 404 includes a depression defined by an inner concave surface 414 in which a first opening 410 is defined. Moreover, the proximal portion 408 includes a depression defined by an inner concave surface 414 in which a second opening 412 is defined. In other embodiments, the depression may be defined by a surface that tapers from the primal-most terminus of the acoustic collector 400 toward the channel defined between the first and second openings 410, 412 or another surface that has a suitable shape for collecting and then guiding sound waves toward the proximal portion 408.

In some embodiments, the medial portion 406 located between the distal and proximal portions 404, 408 acts as a throat segment (or simply “throat”) through which sound waves are directed. In FIG. 4, for example, the width of the channel defined between the first and second openings 410, 412 is widest near the distal and proximal interfaces and narrowest in the medial portion 406. Those skilled in the art will recognize, however, that the dimensions of the channel may vary depending on the form of the inner surface 414 of the tubular body 402.

Moreover, a diaphragm 416 may extends across the first opening 410 of the channel defined through the tubular body 402. As mentioned above, the diaphragm 416 can be used to listen to high-pitch sounds. At a high level, the diaphragm 416 may be representative of a thin sheet of material (e.g., plastic) that vibrates when struck by sound waves that originate in the body. The diagraph 416 may be connected to the distal interface of the tubular body 402 using an adhesive 418 as discussed above.

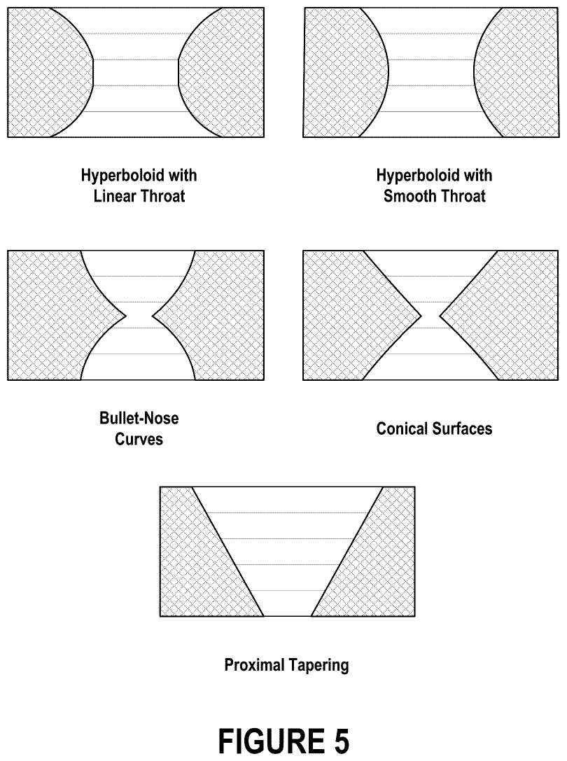

Several examples of different channel geometries are shown in FIG. 5. These channel geometries include hyperboloids with linear throats, hyperboloids with smooth throats, bullet-nose curves, and conical surfaces. Alternatively, the inner surface of the tubular body could be tapered such that the channel narrows toward the proximal interface so as to funnel the sound waves toward the proximal portion.

FIG. 6 is a side view of an acoustic collector 600. Normally, the acoustic collector 600 is wider than it is tall. For example, the width of the acoustic collector may be 5-10 mm as mentioned above. Meanwhile, the length (also called the “height”) of the tubular body 602 is normally 1-5 mm, 2-4 mm, or 2.5-3.5 mm. While the tubular body 602 may be longer than 5 mm in some embodiments, such acoustic collectors can be difficult to manage as those acoustic collectors will extend away from the electronic device to which they are secured as will be further discussed below.

As shown in FIG. 6, the acoustic collector 600 may have an adhesive film 610 (also called an “adhesive layer” or simply “adhesive”) located along at least part of the proximal portion 608. For example, the adhesive 610 may be in the form of an annular ring that extends around the entire periphery of the proximal interface of the tubular body 602. As another example, several “patches” of adhesive 610 may be arranged about the periphery of the proximal interface of the tubular body 602. The adhesive 610 may comprise a temporary or removable adhesive so as to allow the acoustic collector 600 to be easily removed from an electronic device following a recording session. Examples of such adhesives include pressure-sensitive adhesives, sealants, and other non-reactive adhesives. A suitable adhesive may include an elastomer (e.g., an acrylic-based elastomer), EVA, nitrile, silicone rubber, polyurethane, or polymer. In some embodiments, the adhesive 610 further comprises a suitable tackifier. For example, a pressure-sensitive adhesive may be based on an elastomer that is compounded with a rosin ester for tackiness.

In some embodiments, an adhesive 612 is also located along at least part of the distal portion 604. For example, the adhesive 612 may be in the form of an annular ring that extends around the entire periphery of the distal interface of the tubular body 602. As another example, several “patches” of adhesive 612 may be arranged about the periphery of the distal interface of the tubular body 602. Generally, the adhesive 612 is secured, deposited, or otherwise placed on the distal portion 604 such that the diaphragm 614—or at least its central portion—are not covered by the adhesive 612.

In some embodiments, the adhesive 612 along the distal interface comprises a temporary or removable adhesive—much like the adhesive 610 along the proximal interface—such as a pressure-sensitive adhesive, sealant, or another non-reactive adhesive. However, because the distal portion 604 is intended to contact a body while the proximal portion 608 is intended to contact an electronic device, the adhesives 610, 612 may not include the same materials. For example, the adhesive 612 along the distal interface may be non-cytotoxic, hypoallergenic, or resistant to bacterial growth, while the adhesive 610 along the proximal interface may not have such properties. As another example, the adhesive 612 along the distal interface may be less adherent than the adhesive 610 along the proximal interface, since the former may contact the body rather than the electronic device. Specifically the adhesive 612 along the distal interface may be more tacky than adherent, as the primary function of the adhesive 612 is to prevent slippage along the surface of the body.

In other embodiments, the adhesive 612 along the distal interface comprises a permanent adhesive. In such embodiments, rather than contact the body directly, the adhesive 612 may be disposed between the distal interface of the tubular body 602 and a diaphragm 614. When held against the surface of the body, the diaphragm 614 may vibrate when struck by sound waves that originate in the body. The diaphragm 614 allows those sound waves to be more readily directed or collected into the channel defined through the tubular body 602 (and thus guided toward a microphone).

FIG. 7 includes side and overhead views illustrating how an acoustic collector 700 can be secured to an electronic device 702 and then placed against the surface of a body 704. Note that the body could be a human body or an animal body. Initially, an individual may remove a cover from the proximal end of the acoustic collector 700 in order to expose the adhesive disposed along that end. The individual can then secure the exposed proximal end to the electronic device 702. As shown in FIG. 7, the acoustic collector 700 can be secured to the electronic device 702 such that the microphone is located within the bounds of the proximal interface. More specifically, the individual may try to locate the acoustic collector 700 so that the microphone is located near the center of the opening along the proximal interface.

Then, the individual may indicate that she is interested in initiating a recording session. For example, the individual may specify through a computer program executing on the electronic device 700 that she is interested in recording internal sounds originating from within the body 704. To record these internal sounds, the individual may position the electronic device 702 such that the distal end of the acoustic collector 700 contacts the surface of the body 704. As mentioned above, in some embodiments, adhesive is also located along the distal end of the acoustic collector 700, and therefore the individual may remove another cover from the distal end of the acoustic collector 700 in order to expose the adhesive disposed along that end and then secure that end to the surface of the body 704. Generally, the orientation of the electronic device 700 (and thus, the acoustic collector 700) is not relevant so long as contact can be maintained between the distal end of the acoustic collector 700 and the surface of the body 704.

Over the course of the recording session, sound waves that are representative of internal sounds will be collected through the distal interface of the acoustic collector 700. These sound waves will be directed along a channel through the proximal interface of the acoustic collector 700 toward the microphone of the electronic device 702.

At a high level, the acoustic collector 700 serves several purposes. First, the acoustic collector 700 extends the path along which sound waves can be collected by a microphone. Second, the acoustic collector 700 acts similar to the auricle (also called the “pinna”) of the outer ear by channeling sound waves toward a destination (i.e., the microphone). Third, the acoustic collector 700 inhibits the impact of sounds that originate outside of the body 704. These sounds may be referred to as “external sounds.” External sounds generally include a combination of sounds produced by three difference sources: (1) sounds originating from the ambient environment; (2) sounds that leak through the acoustic collector 700; and (3) sounds that penetrate the body 704 under examination. Examples of external sounds include sounds that originate directly from the acoustic collector 700 (e.g., scratching of the tubular body, compressing or stretching of the tubular body) and low-frequency environmental noises that penetrate the acoustic collector 700 or body 704.

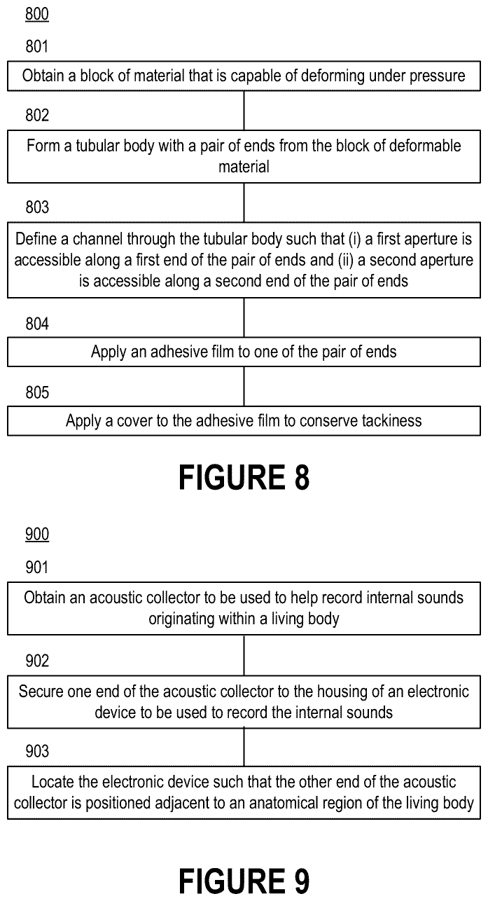

FIG. 8 depicts a flow diagram of a process 800 for manufacturing an acoustic collector. Initially, a manufacturer can obtain a block of a material that is capable of deforming under pressure (step 801). Normally, the deformable material is also capable of reverting to its original form-or at least a close approximation of its original form-upon removal of the pressure. For the purpose of illustration, the material may be described as being acquired in the form of a “block.” However, those skilled in the art will recognize that the process 800 is similarly applicable regardless of the form of the material. In some embodiments, the material is acquired in the form of a roll or tube instead of a block, in which case the manufacturer may perform different steps to manufacture the acoustic collector. Examples of deformable materials include elastomeric materials, sponge materials, foam materials, and the like. Thus, the block may be comprised of an open cell foam that is made from nylon, urethane, latex, or silicone. Evonik VESTAMID® Care ML24 nylon foam, PORON® polyurethane foam, and BISCO® silicone foam are examples of resilient foams that provide high resistance to compression set (i.e., collapse from pressure), as well as high resiliency, vibration damping, and impact absorption.

Thereafter, the manufacturer can form a tubular body with a pair of ends from the block of deformable material (step 802). The tubular body may be no more than 10 mm in width and no more than 5 mm in length. However, the tubular body may be at least 0.05 mm in length. Then, the manufacturer can define a channel through the tubular body such that (i) a first aperture is accessible along a first end of the pair of ends and (ii) a second aperture is accessible along a second end of the pair of ends (step 803). Together, the inner and outer surfaces of the tubular body can take various forms. For example, the tubular body may be in the form of a right circular hollow cylinder (also called a “cylindrical shell”) that is defined by two right circular cylinders that share an axis in common and a pair of ends perpendicular to the common axis. The first aperture (also called the “first opening” of the channel) may be located at a first end of the cylindrical shell, and the second aperture (also called the “second opening” of the channel) may be located at a second end of the cylindrical shell. In some embodiments the first and second apertures have comparable dimensions, while in other embodiments the first and second apertures have different dimensions. Thus, the first aperture may have a different width than the second aperture.

The manufacturer can then apply an adhesive to one of the pair of ends of the tubular body (step 804) and apply a cover to the adhesive to conserve tackiness (step 805). The adhesive may comprise a temporary or removable adhesive so as to allow the acoustic collector to be easily secured to, and then removed from, an electronic device. Examples of such adhesives include pressure-sensitive adhesives, sealants, and other non-reactive adhesives.

Other steps could also be included in the process 800.

For example, the same adhesive or a different adhesive could be applied to the other one of the pair of ends of the tubular body as discussed above. Generally, an adhesive is applied along the end of the tubular body that is to be used as the proximal end. That is, an adhesive film is normally applied along the end of the tubular body that is to be secured to the electronic device. The distal end of the tubular body that is to be positioned against the surface of the body under examination may also include an adhesive, though adhesive may not be necessary to maintain good contact with the surface of the body so long as the acoustic collector is pressed against the surface of the body with sufficient force.

As another example, the manufacturer may apply a coating to the outer surface and/or inner surface of the tubular body to inhibit entry of sound waves into the channel from locations other than the pair of ends of the tubular body. The coating could be used not only to inhibit entry of external sounds, but also to protect against reactions, for example, by the skin from contact with acoustic collectors with tubular bodies comprised of certain materials (e.g., latex). Accordingly, in some embodiments, at least the outer surface of the tubular body could has a coating applied thereto. The coating may comprise wax, rubber, plastic, or the like. Generally, the coating comprises a deformable material such that the tubular body is still permitted to deform when pressure is applied thereto as discussed above.

FIG. 9 depicts a flow diagram of a process 900 for acquiring audio data indicative of sounds internal to a living body. Initially, an individual can obtain an acoustic collector (step 901). The individual could be, for example, a patient who is interested in using the acoustic collector to record her own internal sounds. Alternatively, the individual could be a friend or family member who is interested in recording the internal sounds of another person. While the acoustic collectors described herein could be used by healthcare professionals, those healthcare professionals are generally trained to use sophisticated medical devices, such as electronic and acoustic stethoscopes, that render the acoustic collector largely obsolete.

Thereafter, the individual can secure the acoustic collector to the housing of an electronic device to be used to record the internal sounds (step 902). For example, the individual may remove a cover (also called a “liner”) along one end of the acoustic collector to expose an adhesive and then attach the acoustic collector to the housing of the electronic device using the adhesive. As discussed above, the acoustic collector may be secured to the housing of the electronic device such that the one end is positioned adjacent to a hole in the housing through which sound waves can travel to reach a microphone.

The individual can then locate the electronic device such that the other end is positioned adjacent to an anatomical region of the living body (step 903). For example, the individual may hold the electronic device so that the other end is directly adjacent to the skin of the living body in the anatomical region. Generally, the anatomical region depends upon the internal sounds that are of interest to the individual. For example, if the individual is interested in recording circulatory or respiratory sounds, then she may hold the other end of the acoustic collector against the chest region. As another example, if the individual is interested in recording gastrointestinal sounds, then she may hold the other end of the acoustic collector against the abdominal region.

Note that the individual may be able to initiate the recording session at any point during the process 900. For example, the individual may prompt the electronic device to begin recording after the acoustic collector has been secured to the housing of the electronic device, or the individual may prompt the electronic device to begin recording after the acoustic collector has been positioned adjacent to the anatomical region of the living body. Normally, the individual accomplishes this by interacting with a computer program executing on the electronic device. However, the computer program could be configured to automatically begin recording, for example, upon detecting sounds that are representative of internal sounds.

Unless contrary to physical possibility, it is envisioned that the steps described above may be performed in various sequences and combinations. For example, multiple instances of the process 900 of FIG. 9 may be performed in order to generate multiple recordings corresponding to the same anatomical region or different anatomical regions.

### Overview of Electronic Stethoscope System

As discussed above, acoustic collectors could be employed—on their own—to facilitate the collecting of sound waves originating from within a body and recording of those sound waves by a microphone of an electronic device. However, acoustic collectors could also be used in the input units of an electronic stethoscope system.

As further discussed below, an electronic stethoscope system may include one or more input units that are connected to a hub unit. Each input unit may have a conical resonator cavity (also called a “conical resonator” or “resonator cavity”) that is designed to direct sound waves toward at least one microphone that is configured to produce audio data indicative of internal sounds originating inside the living body. These microphones may be referred to as “auscultation microphones.” Moreover, each input unit may include at least one microphone that is configured to produce audio data indicative of external sounds originating outside the living body. These microphones may be referred to as “ambient microphones” or “environmental microphones.” For the purpose of illustration, an “ambient microphone” may be described as being capable of producing audio data indicative of “ambient sounds.” However, these “ambient sounds” generally include a combination of external sounds as discussed above.

There are several advantages to separately recording internal and external sounds. Notably, the internal sounds can be electronically amplified while the external sounds can be electronically dampened, attenuated, or filtered. Thus, the electronic stethoscope system may address the faint sounds originating from within the living body under examination by manipulating the audio data indicative of the internal and external sounds. Manipulation may result in undesirable digital artifacts that make it more difficult to interpret the internal sounds, however. By using an acoustic collector, the quality of the audio data indicative of the internal sounds can be improved without relying on manipulation (e.g., dampening, attenuating, or filtering) of the underlying signal.

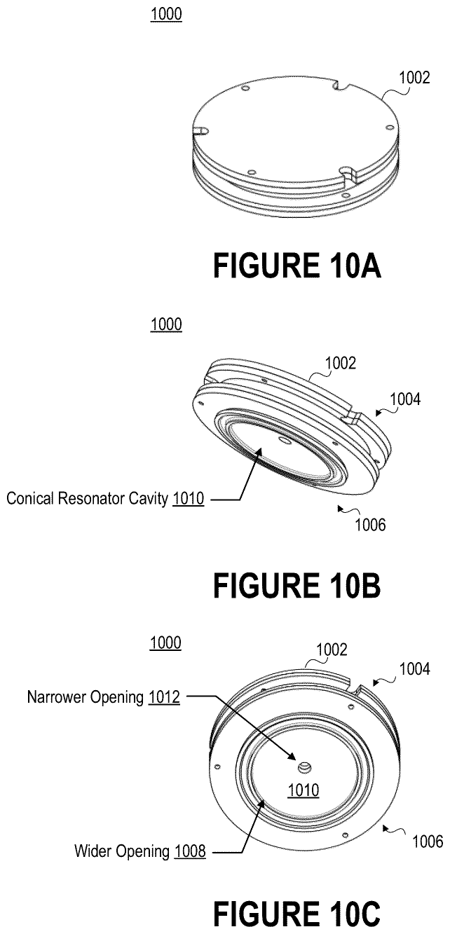

FIG. 10A includes a top perspective view of an input unit 1000 for an electronic stethoscope system. For convenience, the input unit 1000 may be referred to as a “stethoscope patch,” even though the input unit may only include a subset of the components necessary for auscultation. The input unit 1000 may also be referred to as a “chestpiece” since it will often be affixed to the chest of a body. However, those skilled in the art will recognize that the input unit 1000 may be affixed to other parts of the body as well (e.g., the neck, abdomen, or back).

As further described below, the input unit 1000 can collect sound waves that are representative of internal sounds, convert the sound waves into an electrical signal, and then digitize the electrical signal (e.g., for easier transmission, to ensure higher fidelity, etc.). The input unit 1000 can include a structural body 1002 that is comprised of a rigid material. Normally, the structural body 1002 is comprised of metal, such as stainless steel, aluminum, titanium, or a suitable metal alloy. To make the structural body 1002, molten metal will typically be die-cast and then either machined or extruded into the appropriate form.

In some embodiments, the input unit 1000 includes a casing that inhibits exposure of the structural body 1002 to the ambient environment. For example, the casing may prevent contamination, improve cleanability, improve clarity, etc. Generally, the casing encapsulates substantially all of the structural body 1002 except for the conical resonator cavity disposed along its bottom side. The conical resonator cavity is described in greater depth below with respect to FIGS. 10B-C. The casing may be comprised of silicon rubber, polypropylene, polyethylene, or any other suitable material. Moreover, in some embodiments, the casing includes an additive whose presence limits microbial growth, ultraviolet (“UV”) degradation, etc.

FIGS. 10B-C include bottom perspective views of the input unit 1000, which includes a structural body 1002 having a distal portion 1004 and a proximal portion 1006. To initiate an auscultation procedure, an individual (e.g., a healthcare professional, such as a physician or nurse) can secure the proximal portion 1006 of the input unit 1000 against the surface of a body under examination. The proximal portion 1006 of the input unit 1000 can include the wider opening 1008 of a conical resonator cavity 1010. The conical resonator cavity 1010 may be designed to direct sound waves collected through the wider opening 1008 toward a narrower opening 1012, which may lead to an auscultation microphone. Conventionally, the wider opening 1008 is approximately 30-50 mm, 35-45 mm, or 38-40 mm. However, because the input unit 1000 described here may have improved isolation of internal sounds, smaller conical resonator cavities may be used. For example, in some embodiments, the wider opening 1008 is less than 30 mm, 20 mm, or 10 mm. Thus, the input units described herein may be able to support a wide variety of conical resonator cavities having different sizes, designed for different applications, etc.

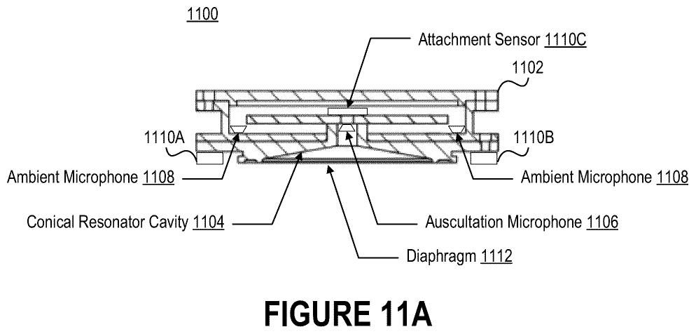

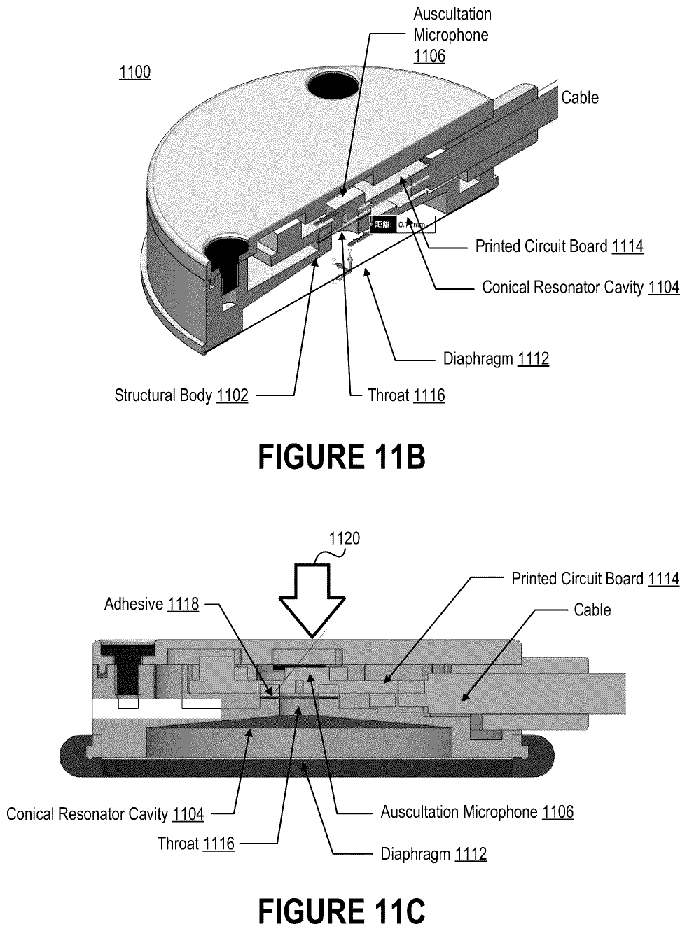

FIG. 11A includes a cross-sectional side view of an input unit 1100 for an electronic stethoscope system that does not include an acoustic collector. Often, the input unit 1100 includes a structural body 1102 having an interior cavity defined therein. The structural body 1102 of the input unit 1100 may have a conical resonator cavity 1104 designed to direct sound waves toward a microphone residing within the interior cavity. In some embodiments, a diaphragm 1112 (also referred to as a “vibration film”) extends across the wider opening (also referred to as the “outer opening”) of the conical resonator cavity 1104. The diaphragm 1112 can be used to detect vibrations induced by sound waves received through the conical resonator cavity 1104. The diaphragm 1112 can be formed from a thin plastic disk comprised of an epoxy-fiberglass compound or glass fibers.

To improve the clarity of sound waves collected by the conical resonator cavity 1104, the input unit 1100 may be designed to simultaneously monitor sounds originating from different locations. For example, the input unit 1100 may be designed to simultaneously monitor sounds originating from within a body under examination and sounds originating from the ambient environment. Thus, the input unit 1100 may include at least one microphone 1106 (referred to as an “auscultation microphone”) configured to produce audio data indicative of internal sounds and at least one microphone 1108 (referred to as an “ambient microphone”) configured to produce audio data indicative of ambient sounds. Each auscultation and ambient microphone may include a transducer able to convert sound waves into an electrical signal. Thereafter, the electrical signals produced by the auscultation and ambient microphones 1106, 1108 may be digitized prior to transmission to a hub unit. Digitization enables the hub unit to readily clock or synchronize the signals received from multiple input units. Digitization may also ensure that the signals received by the hub unit from an input unit have a higher fidelity than would otherwise be possible.

These microphones may be omnidirectional microphones designed to pick up sound from all directions or directional microphones designed to pick up sounds coming from a specific direction. For example, the input unit 1100 may include auscultation microphone(s) 1106 oriented to pick up sounds originating from a space adjacent to the outer opening of the conical resonator cavity 1104. In such embodiments, the ambient microphone(s) 1108 may be omnidirectional or directional microphones. As another example, a set of ambient microphones 1108 could be equally spaced within the structural body 1102 of the input unit 1100 to form a phased array able to capture highly directional ambient sounds to reduce noise and interference. Accordingly, the auscultation microphone(s) 1106 may be arranged to focus on the path of incoming internal sounds (also referred to as the “auscultation path”), while the ambient microphone(s) 1108 may be arranged to focus on the paths of incoming ambient sounds (also referred to as the “ambient paths”).

Conventionally, electronic stethoscopes subjected electrical signals indicative of sound waves to digital signal processing (“DSP”) algorithms that were responsible for filtering undesirable artifacts. However, such action could suppress nearly all of the sound within certain frequency ranges (e.g., 100-800 Hz), thereby greatly distorting internal sounds of interest (e.g., those corresponding to inhalations, exhalations, or heartbeats). Here, however, a processor can employ an active noise cancellation algorithm that separately examines the audio data generated by the auscultation microphone(s) 1106 and the audio data generated by the ambient microphone(s) 1108. More specifically, the processor may parse the audio data generated by the ambient microphone(s) 1108 to determine how, if at all, the audio data generated by the auscultation microphone(s) 1106 should be modified. For example, the processor may discover that certain digital features should be amplified (e.g., because they correspond to internal sounds), diminished (e.g., because they correspond to ambient sounds), or removed entirely (e.g., because they represent noise). Such a technique can be used to improve the clarity, detail, and quality of sound recorded by the input unit 1100. For example, application of the noise cancellation algorithm may be an integral part of the denoising process employed by an electronic stethoscope system that includes at least one input unit 1100.

For privacy purposes, neither the auscultation microphone(s) 1106 nor the ambient microphone(s) 1108 may be permitted to record while the conical resonator 1104 is directed away from the body. Thus, in some embodiments, the auscultation microphone(s) 1106 and/or the ambient microphone(s) 1108 do not begin recording until the input unit 1100 is attached to the body. In such embodiments, the input unit 1100 may include one or more attachment sensors 1110A-C that are responsible for determining whether the structural body 1102 has been properly secured to the surface of the body.

The input unit 1100 could include any subset of the attachment sensors shown here. For example, in some embodiments, the input unit 1100 includes only attachment sensors 1110A-B, which are positioned near the wider opening of the conical resonator cavity 1104. As another example, in some embodiments, the input unit 1100 includes only attachment sensor 1110C, which is positioned near the narrower opening (also called the “inner opening”) of the conical resonator cavity 1104. Moreover, the input unit 1100 may include different types of attachment sensors. For example, attachment sensor 1110A may be an optical proximity sensor designed to emit light (e.g., infrared light) through the conical resonator cavity 1104 and then determine, based on the light reflected back into the conical resonator cavity 1104, the distance between the input unit 1100 and the surface of the body. As another example, attachment sensors 1110A-C may be audio sensors designed to determine, with the assistance of an algorithm programmed to determine the drop-off of a high-frequency signal, whether the structural body 1102 is securely sealed against the surface of the body based on the presence of environmental noise. As another example, attachment sensors 1110A-B may be pressure sensors designed to determine whether the structural body 1102 is securely sealed against the surface of the body based on the amount of applied pressure. Some embodiments of the input unit 1100 include each of these different types of attachment sensors. By considering the output of these attachment sensor(s) 1110A-C in combination with the aforementioned active noise cancellation algorithm, a processor may be able to dynamically determine the adhesion state. That is, the processor may be able to determine whether the input unit 1100 has formed a seal against the body based on the output of these attachment sensors 1110A-C.

FIG. 11B includes a cross-sectional perspective view of the input unit 1100, while FIG. 11C includes a cross-sectional side view of the input unit 1100. As shown in FIGS. 11B-C, an auscultation microphone 1106-which may be positioned near the inner opening of the conical resonator cavity 1104 so that sound waves collected through the outer opening can be “funneled” toward the auscultation microphone 1106 via a throat 1116-can be mounted to, or made accessible through, a printed circuit board 1114. Such an arrangement may cause the auscultation microphone 1106 to be about 0.5-1.0 mm (and generally 0.6-0.8 mm) away from the inner opening of the conical resonator cavity 1104. Note that, in some embodiments, an adhesive 1118 may be situated between the printed circuit board 1114 and the portion of the structural body 1102 that defines the conical resonator cavity 1104. However, the adhesive 1118 is generally quite thin (e.g., less than 0.1 mm), and therefore does not “lengthen” the throat 1116 or act as a cushion between the printed circuit board 1114 and the portion of the structural body 1102 that defines the conical resonator cavity 1104.

The height of the adhesive 1118 and throat 1116—as measured from the auscultation microphone 1106 to the inner opening of the conical resonator cavity 1104—will not meaningfully change even if sizable force 1120 is applied to the input unit 1100. Therefore, the input unit 1000 can be said to have a relatively high compression ratio. A higher compression ratio generally corresponds to noisier signals, and therefore lowering the compression ratio is desirable. Moreover, the printed circuit board 1114 may be connected, via the adhesive 1118, to the portion of the structural body 1102 that defines, or corresponds to (e.g., complements), the periphery of the conical resonator cavity 1104 as shown in FIG. 11B, and such a design can result in undesirable noise. For example, if the structural body 1102 is dragged across the surface of a living body, these vibrations can transfer from the structural body 1102 to the printed circuit board 1114 (and therefore, the auscultation microphone 1106) via the adhesive 1118.

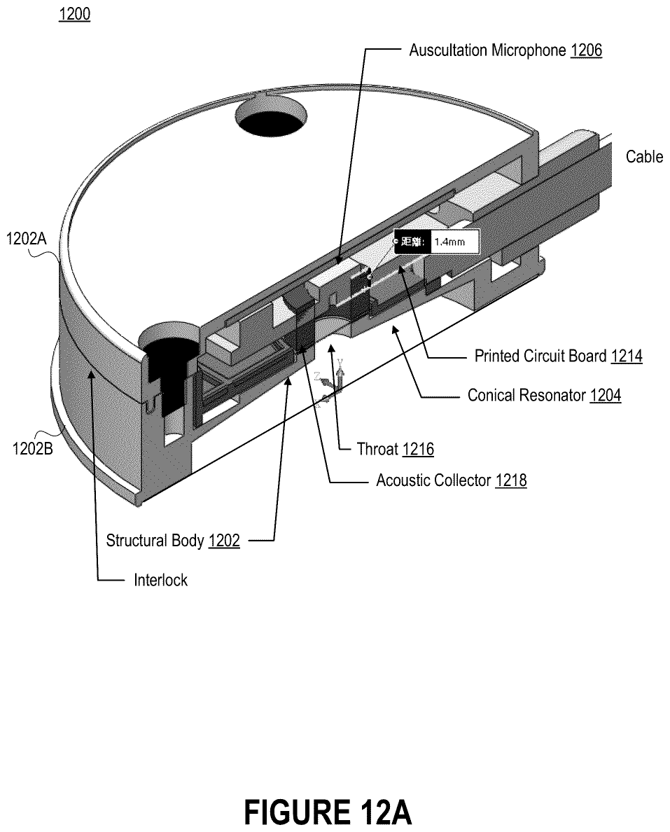

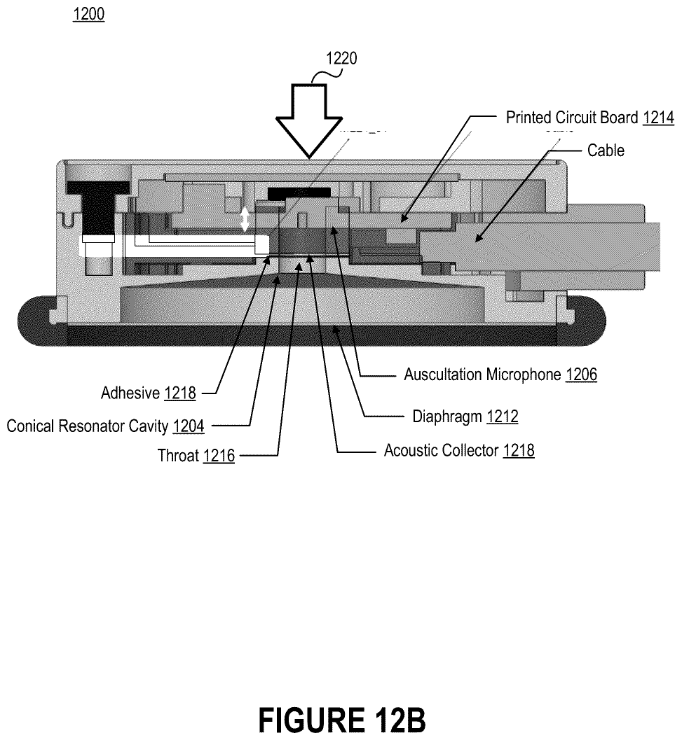

FIG. 12A includes a cross-sectional perspective view of an input unit 1200 that does include an acoustic collector 1218, and FIG. 12B includes a cross-sectional side view of the input unit 1200. Note that except for the addition of the acoustic collector 1218, the input unit 1200 may otherwise be similar to the input unit 1100 of FIGS. 11A-C. Accordingly, the input unit 1200 may include an auscultation microphone 1206 that is positioned near the inner opening of a conical resonator cavity 1204 so that sound waves collected through the outer opening can be “funneled” toward the auscultation microphone 1206. The auscultation microphone 1206 can be mounted to, or made accessible through, a printed circuit board 1214.

Here, an acoustic collector 1218 is situated between the printed circuit board 1214 and the portion of the structural body 1202 that defines, or corresponds to (e.g., complements), the periphery of the conical resonator cavity 1204. With the addition of the acoustic collector 1218, the throat 1216 is “lengthened.” For example, the acoustic collector 1218 may have a thickness of 2.0-4.0 mm. By “lengthening” the throat 1216, a lower compression ratio can be achieved, resulting in less noisy signals.

Moreover, the acoustic collector 1218 may include a deformable material as mentioned above. For example, the acoustic collector 1218 may comprise a deformable material, such as an open cell foam or a closed cell foam made of nylon, urethane, latex, or silicone. Evonik VESTAMID® Care ML24 nylon foam, PORON® polyurethane foam, and BISCO® silicone foam are examples of suitable foams. Embodiments of the acoustic collector 1218 could have a density of less than 0.8 grams per cubic centimeter (“g/cm³”), 0.6 g/cm³, or 0.4 g/cm³.

Because the acoustic collector 1218 comprises a deformable material, the “length” of the throat 1216 can vary when force 1220 is applied to the input unit 1200. Specifically, the height of the acoustic collector 1218—and therefore, the distance between the auscultation microphone 1206 and the inner opening of the conical resonator cavity 1204—can decrease when the force 1220 is applied to the input unit. For example, the acoustic collector 1218 may shrink, compress, or otherwise deform such that the acoustic collector 1218 has a compressed thickness of 1.0-2.0 mm, 1.2-1.8 mm, or 1.4-1.6 mm. As mentioned above, deformation along the longitudinal axis can actually stabilize the acoustic collector 1218, providing rigidity and strengthening the isolation of the sound waves traveling through the channel, thereby resulting in improved auscultation.

In embodiments where the acoustic collector 1218 is porous material, situating the acoustic collector 1218 between—for example, directly adjacent to—the printed circuit board 1214 and the structural body 1202 can also reduce noise. At a high level, the acoustic collector 1218 may act as an acoustic insulator within the structural body 1202 of the input unit 1200. Not only can the acoustic collector 1218 create a more acoustically accommodating throat 1216, but the acoustic collector 1218 can prevent, inhibit, or otherwise limit undesirable noise. For example, if the structural body 1202 is dragged across the surface of a living body, these vibrations can transfer from the acoustic collector 1218 rather than directly to the printed circuit board 1214 (and therefore, the auscultation microphone 1206).

As discussed above with reference to FIG. 6, the acoustic collector 1218 may have adhesive applied to its distal interface to facilitate securement to the structural body 1202 and/or its proximal interface to facilitate securement to the printed circuit board 1214. Adhesive may be applied to the proximal interface before the acoustic collector 1218 is affixed to the bottom side of the printed circuit board 1214, and then adhesive may be applied to the distal interface before the acoustic collector 1218 is affixed to the inner surface of the structural body 1202. Alternatively, adhesive may be applied to the distal interface before the acoustic collector 1218 is affixed to the inner surface of the structural body 1202, and then adhesive may be applied to the proximal interface before the acoustic collector 1218 is affixed to the bottom side of the printed circuit board 1218. In some embodiments, adhesive is only applied along the distal interface of the acoustic collector 1218, as shown in FIG. 12B and indicated with reference numeral 1218. In some embodiments, the adhesive 1218 is in the form of a double-sided tape, for example, comprising a polyethylene terephthalate (“PET”) backing and an acrylic adhesive. In other embodiments, the adhesive 1218 is in the form of a gel, for example, that includes a heat-, ultraviolet-, or chemically-activated resin.

As shown in FIGS. 12A-B, the structural body 1202 may comprise a first portion 1202A and a second portion 1202B that interlock along the periphery of the input unit 1200. Segmenting the structural body 1202 provides flexibility in how and when the acoustic collector 1218 is installed within the input unit 1200.

Accordingly, the input unit 1200 may comprise a printed circuit board 1214 with a first side and a second side on which an auscultation microphone 1206 is mounted, a structural body that includes (i) a first section and (ii) a second section with an exterior surface that defines a resonator cavity through which sound waves are guided toward an auscultation microphone when the structural body is placed against a surface of a living body, and an acoustic collector with a tubular body comprised of a deformable material that is situated between the second side of the printed circuit board and the second section of the structural body. The tubular body may be cylindrical in form, so as to encircle the acoustic microphone while making contact with the second side of the printed circuit board.

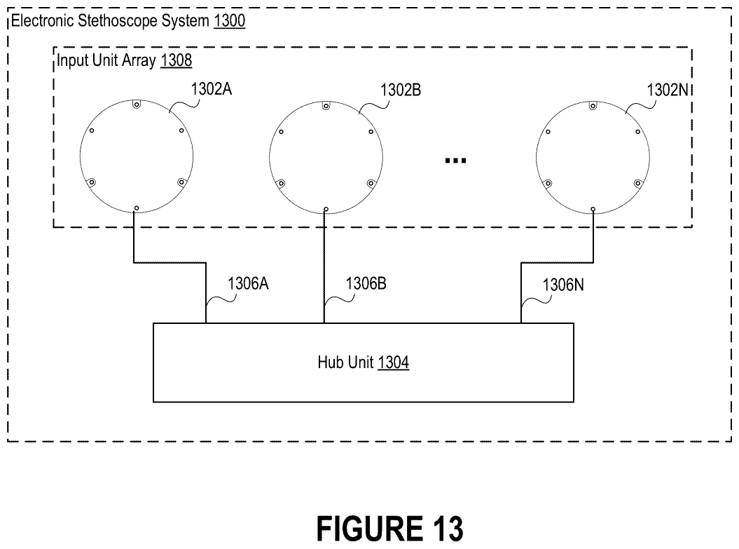

FIG. 13 illustrates how one or more input units 1302A-N can be connected to a hub unit 1304 to form an electronic stethoscope system 1300. In some embodiments, multiple input units are connected to the hub unit 1304. For example, the electronic stethoscope system 1300 may include four input units, six input units, or eight input units. Generally, the electronic stethoscope system 1300 will include at least six input units. Electronic stethoscope systems that have multiple input units may be referred to as “multi-channel stethoscopes.” In other embodiments, only one input unit is connected to the hub unit 1304. For example, a single input unit may be moved across the body in such a manner as to simulate an array of multiple input units. Electronic stethoscope systems having one input unit may be referred to as “single-channel stethoscopes.”

As shown in FIG. 13, each input unit 1302A-N can be connected to the hub unit 1304 via a corresponding cable 1306A-N. Generally, the transmission path formed between each input unit 1302A-N and the hub unit 1304 via the corresponding cable 1306A-N is designed to be substantially free of interference. For example, electronic signals may be digitized by the input units 1302A-N prior to transmission to the hub unit 1304, and signal fidelity may be ensured by prohibiting the generation/contamination of electromagnetic noise. Examples of cables include ribbon cables, coaxial cables, Universal Serial Bus (“USB”) cables, High-Definition Multimedia Interface (“HDMI”) cables, RJ45 ethernet cables, and any other cable suitable for conveying a digital signal. Each cable includes a first end connected to the hub unit 1304 (e.g., via a physical port) and a second end connected to the corresponding input unit (e.g., via a physical port). Accordingly, each input unit 1302A-N may include a single physical port, and the hub unit 1304 may include multiple physical ports. Alternatively, a single cable may be used to connect all of the input units 1302A-N to the hub unit 1304. In such embodiments, the cable may include a first end capable of interfacing with the hub unit 1304 and a series of second ends, each of which is capable of interfacing with a single input unit. Such a cable may be referred to, for example, as a “one-to-two cable,” “one-to-four cable,” or “one-to-six cable” based on the number of second ends.

When all of the input units 1302A-N connected to the hub unit 1304 are in an auscultation mode, the electronic stethoscope system 1300 can employ an adaptive gain control algorithm programmed to compare internal sounds to ambient sounds. The adaptive gain control algorithm may analyze a target auscultation sound (e.g., normal breathing, wheezing, crackling, etc.) to judge whether an adequate sound level has been achieved. For example, the adaptive gain control algorithm may determine whether the sound level exceeds a predetermined threshold. The adaptive gain control algorithm may be designed to achieve gain control of up to 100 times (e.g., in two different stages). The gain level may be adaptively adjusted based on the number of input units in the input unit array 1308, as well as the level of sound recorded by the auscultation microphone(s) in each input unit. In some embodiments, the adaptive gain control algorithm is programmed for deployment as part of a feedback loop. Thus, the adaptive gain control algorithm may apply gain to audio recorded by an input unit, determine whether the audio exceeds a preprogrammed intensity threshold, and dynamically determine whether additional gain is necessary based on the determination.

Because the electronic stethoscope system 1300 can deploy the adaptive gain control algorithm during a postprocessing procedure, the input unit array 1308 may be permitted to collect information regarding a wide range of sounds caused by the heart, lungs, etc. Because the input units 1302A-N in the input unit array 1308 can be placed in different anatomical positions along the surface of the body (or on an entirely different body), different biometric characteristics (e.g., respiratory rate, heart rate, or degree of wheezing, crackling, etc.) can be simultaneously monitored by the electronic stethoscope system 1300.

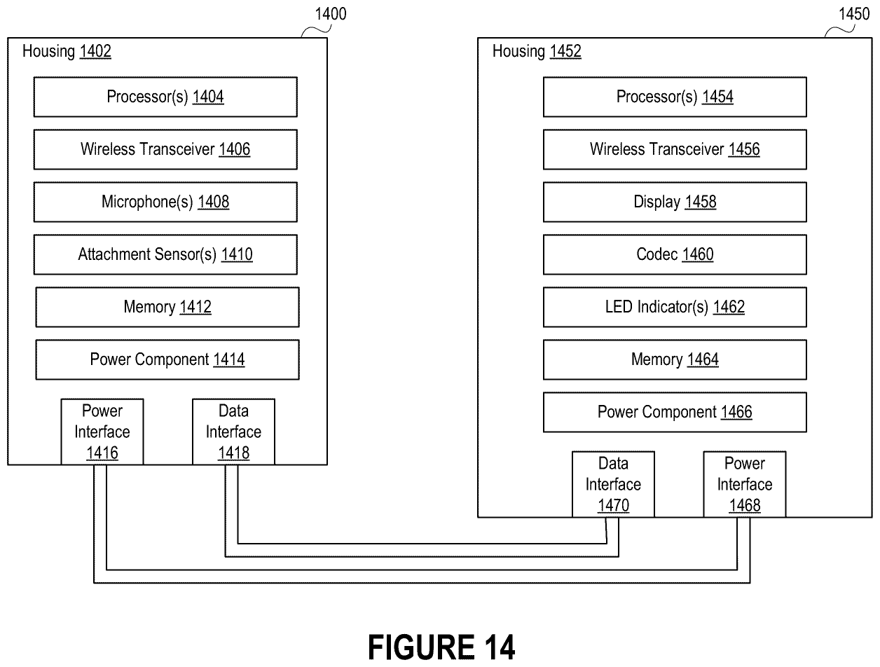

FIG. 14 is a high-level block diagram illustrating exemplary components of an input unit 1400 and a hub unit 1450 of an electronic stethoscope system. Embodiments of the input unit 1400 and the hub unit 1450 can include any subset of the components shown in FIG. 14, as well as additional components not illustrated here. For example, the input unit 1400 may include a biometric sensor capable of monitoring a biometric characteristic of the body, such as perspiration (e.g., based on skin humidity), temperature, etc. Additionally or alternatively, the biometric sensor may be designed to monitor a breathing pattern (also referred to as a “respiratory pattern”), record electrical activity of the heart, etc. As another example, the input unit 1400 may include an inertial measurement unit (“IMU”) capable of generating data from which gesture, orientation, or position can be derived. An IMU is an electronic component designed to measure the force, angular rate, inclination, and/or magnetic field of an object. Generally, IMUs include accelerometer(s), gyroscope(s), magnetometer(s), or any combination thereof.

The input unit 1400 can include one or more processors 1404, a wireless transceiver 1406, one or more microphones 1408, one or more attachment sensors 1410, a memory 1412, and/or a power component 1414 that is electrically coupled to a power interface 1416. These components may reside within a housing 1402 (also called a “structural body”).

As noted above, the microphone(s) 1408 can convert acoustic sound waves into an electrical signal. The microphone(s) 1408 may include auscultation microphone(s) configured to produce audio data indicative of internal sounds, ambient microphone(s) configured to produce audio data indicative of ambient sounds, or any combination thereof. Audio data representative of values of the electrical signal can be stored, at least temporarily, in the memory 1412. In some embodiments, the processor(s) 1404 process the audio data prior to transmission downstream to the hub unit 1450. For example, the processor(s) 1404 may apply algorithms designed for digital signal processing, denoising, gain control, noise cancellation, artifact removal, feature identification, etc. In other embodiments, minimal processing is performed by the processor(s) 1404 prior to transmission downstream to the hub unit 1450. For example, the processor(s) 1404 may simply append metadata to the audio data that specifies the identity of the input unit 1400 or examine metadata already added to the audio data by the microphone(s) 1408.

In some embodiments, the input unit 1400 and the hub unit 1450 transmit data between one another via a cable connected between corresponding data interfaces 1418, 1470. For example, audio data generated by the microphone(s) 1408 may be forwarded to the data interface 1418 of the input unit 1400 for transmission to the data interface 1470 of the hub unit 1450. Alternatively, the data interface 1470 may be part of the wireless transceiver 1456. The wireless transceiver 1406 could be configured to automatically establish a wireless connection with the wireless transceiver 1456 of the hub unit 1450. The wireless transceivers 1406, 1456 may communicate with one another via a bidirectional communication protocol, such as Near Field Communication (“NFC”), wireless USB, Bluetooth®, Wi-Fi®, a cellular data protocol (e.g., LTE, 3G, 4G, or 5G), or a proprietary point-to-point protocol.

The input unit 1400 may include a power component 1414 that is able to provide power to the other components residing within the housing 1402, as necessary. Similarly, the hub unit 1450 can include a power component 1466 that is able to provide power to the other components residing within the housing 1452. Examples of power components include rechargeable lithium-ion (“Li-Ion”) batteries, rechargeable nickel-metal hydride (“NiMH”) batteries, rechargeable nickel-cadmium (“NiCad”) batteries, etc. In some embodiments, the input unit 1400 does not include a dedicated power component, and thus must receive power from the hub unit 1450. A cable designed to facilitate the transmission of power (e.g., via a physical connection of electrical contacts) may be connected between a power interface 1416 of the input unit 1400 and a power interface 1468 of the hub unit 1450.

The power channel (i.e., the channel between power interface 1416 and power interface 1468) and the data channel (i.e., the channel between data interface 1418 and data interface 1470) have been shown as separate channels for the purpose of illustration only. Those skilled in the art will recognize that these channels could be included in the same cable. Thus, a single cable capable of carrying data and power may be coupled between the input unit 1400 and the hub unit 1450.

The hub unit 1450 can include one or more processors 1454, a wireless transceiver 1456, a display 1458, a codec 1460, one or more light-emitting diode (“LED”) indicators 1462, a memory 1464, and a power component 1466. These components may reside within a housing 1452 (also called a “structural body”). As noted above, embodiments of the hub unit 1450 may include any subset of these components, as well as additional components not shown here.

As shown in FIG. 14, embodiments of the hub unit 1450 may include a display 1458 for presenting information such as the respiratory status or heart rate of an individual under examination, a network connectivity status, a power connectivity status, a connectivity status for the input unit 1400, etc. The display 1458 may be controlled via tactile input mechanisms (e.g., buttons accessible along the surface of the housing 1452), audio input mechanisms (e.g., microphones), and the like. As another example, some embodiments of the hub unit 1450 include LED indicator(s) 1462 for operation guidance rather than the display 1458. In such embodiments, the LED indicator(s) 1462 may convey information similar to that presented by the display 1458. As another example, some embodiments of the hub unit 1450 include a display 1458 and LED indicator(s) 1462.

Upon receiving audio data representative of the electrical signal generated by the microphone(s) 1408 of the input unit 1400, the hub unit 1450 may provide the audio data to a codec 1460 that is responsible for decoding the incoming data. The codec 1460 may, for example, decode the audio data (e.g., by reversing encoding applied by the input unit 400) in preparation for editing, processing, etc. The codec 1460 may be designed to sequentially or simultaneously process audio data generated by the auscultation microphone(s) in the input unit 1400 and audio data generated by the ambient microphone(s) in the input unit 1400.

Thereafter, the processor(s) 1454 can process the audio data. Much like the processor(s) 1404 of the input unit 1400, the processor(s) 1454 of the hub unit 1450 may apply algorithms designed for digital signal processing, denoising, gain control, noise cancellation, artifact removal, feature identification, etc. Some of these algorithms may not be necessary if already applied by the processor(s) 1404 of the input unit 1400. For example, in some embodiments the processor(s) 1454 of the hub unit 1450 apply algorithm(s) to discover diagnostically relevant features in the audio data, while in other embodiments such action may not be necessary if the processor(s) 1404 of the input unit 1400 have already discovered the diagnostically relevant features. Alternatively, the hub unit 1450 may forward the audio data to a destination (e.g., a diagnostic platform running on a computing device or decentralized system) for analysis, as further discussed below. Generally, a diagnostically relevant feature will correspond to a pattern of values in the audio data matching a predetermined pattern-defining parameter. As another example, in some embodiments the processor(s) 1454 of the hub unit 1450 apply algorithms to reduce noise in the audio data to improve the signal-to-noise (“SNR”) ratio, while in other embodiments these algorithms are applied by the processor(s) 1404 of the input unit 1400.

In addition to the power interface 1468, the hub unit 1450 may include a power port. The power port (also referred to as a “power jack”) enables the hub unit 1450 to be physically connected to a power source (e.g., an electrical outlet). The power port may be capable of interfacing with different connector types (e.g., C13, C15, C19). Additionally or alternatively, the hub unit 1450 may include a power receiver that has an integrated circuit (also referred to as a “chip”) able to wirelessly receive power from an external source. Similarly, the input unit 1400 may include a power receiver that has a chip able to wirelessly receive power from an external source, for example, if the input unit 1400 and hub unit 1450 are not physically connected to one another via a cable. The power receiver may be configured to receive power transmitted in accordance with the Qi standard developed by the Wireless Power Consortium or some other wireless power standard.

In some embodiments, the housing 1452 of the hub unit 1450 includes an audio port. The audio port (also referred to as an “audio jack”) is a receptacle that can be used to transmit signals, such as audio, to an appropriate plug of an attachment, such as headphones. An audio port typically includes one, two, three, or four contacts that enable audio signals to be readily transmitted when an appropriate plug is inserted into the audio port. For example, most headphones include a plug designed for a 3.5-mm audio port. Additionally or alternatively, the wireless transceiver 1456 of the hub unit 1450 may be able to transmit audio signals directly to wireless headphones (e.g., via NFC, Wireless USB, Bluetooth, etc.).

As noted above, the processor(s) 1404 of the input unit 1400 and/or the processor(s) 1454 of the hub unit 1450 can apply a variety of algorithms to support different functionalities. Examples of such functionalities include attenuation of lost data packets in the audio data, noise-dependent volume control, dynamic range compression, automatic gain control, equalization, noise suppression, and acoustic echo cancellation. Each functionality may correspond to a separate module residing in a memory (e.g., memory 1412 of the input unit 1400 or memory 1464 of the hub unit 1450). Thus, the input unit 1400 and/or the hub unit 1450 may include an attenuation module, a volume control module, a compression module, a gain control module, an equalization module, a noise suppression module, an echo cancellation module, or any combination thereof.

Note that, in some embodiments, the input unit 1400 is configured to transmit audio data generated by the microphone(s) 1408 directly to a destination other than the hub unit 1450. For example, the input unit 400 may forward the audio data to the wireless transceiver 1406 for transmission to a computing device on which is executing a computer program that is responsible for analyzing the audio data. The audio data may be transmitted to the computing device instead of, or in addition to, the hub unit 1450. If the audio data is forwarded to the computing device in addition to the hub unit 1450, then the input unit 1400 may generate a duplicate copy of the audio data and then forward those separate copies onward (e.g., to the wireless transceiver 1406 for transmission to the computing device, to the data interface 1418 for transmission to the hub unit 1450).

Additional information on electronic stethoscope systems can be found in U.S. Pat. No. 10,555,717, which is incorporated by reference herein in its entirety.

### Remarks

The foregoing description of various embodiments of the technology has been provided for the purposes of illustration and description. It is not intended to be exhaustive or to limit the claimed subject matter to the precise forms disclosed.

Many modifications and variation will be apparent to those skilled in the art. Embodiments were chosen and described in order to best describe the principles of the technology and its practical applications, thereby enabling others skilled in the relevant art to understand the claimed subject matter, the various embodiments, and the various modifications that are suited to the particular uses contemplated.

What is claimed is:

1. An apparatus for guiding sounds toward a destination, the apparatus comprising:
a tubular body that includes—
a distal interface through which sound waves corresponding to sounds internal to a living body are collected,
a proximal interface through which the sound waves are presented to a microphone of an electronic device to which the tubular body is connected, and
a channel defined by an inner surface of the tubular body extending between the distal and proximal interfaces,
wherein the channel allows the sound waves to travel from the distal interface to the proximal interface.

2. The apparatus of claim 1, wherein the tubular body is comprised of a deformable material capable of deforming from an original form under pressure and regaining the original form upon removal of the pressure.

3. The apparatus of claim 2, wherein the deformable material is urethane foam.

4. The apparatus of claim 1, wherein the inner surface is tapered such that the channel narrows toward the proximal interface so as to funnel the sound waves toward the proximal interface.

5. The apparatus of claim 1, wherein the distal interface has a concave surface that is representative of a recess at which to collect the sound waves, and wherein the proximal interface has a concave surface that is representative of a recess at which to present the sound waves.

6. The apparatus of claim 1, further comprising:
a diaphragm that vibrates when struck by the sound waves; and
an adhesive film that secures the diaphragm to the distal interface of the tubular body.

7. The apparatus of claim 1, wherein a diameter of the channel at the distal interface is between 2 millimeters (mm) and 7 mm, and wherein a diameter of the channel at the proximal interface is between 2 mm and 7 mm.

8. The apparatus of claim 7, wherein a diameter of the tubular body is between 5 mm and 10 mm.

9. The apparatus of claim 1, wherein a length of the tubular body is no more than 5 mm.

10. The apparatus of claim 1, further comprising:
an adhesive film that secures a proximal end of the tubular body to the electronic device.

11. The apparatus of claim 10, wherein the adhesive film comprises a temporary adhesive so as to allow the tubular body to be detached from the electronic device.

12. A method for manufacturing an acoustic collection device, the method comprising:
acquiring a block of deformable material that is capable of deforming under pressure and reverting to an original form upon removal of the pressure;
forming a tubular body with a pair of ends from the block of deformable material,
wherein the tubular body is no more than 10 millimeters (mm) in width and no more than 5 mm in length; and
defining a channel through the tubular body such that (i) a first aperture is accessible along a first end of the pair of ends and (ii) a second aperture is accessible along a second end of the pair of ends.

13. The method of claim 12, wherein the tubular body is in the form of a right circular hollow cylinder.

14. The method of claim 12, wherein the deformable material is an open cell foam comprised of urethane, latex, or silicone.

15. The method of claim 12, further comprising:
applying an adhesive film to one of the pair of ends of the tubular body.

16. The method of claim 12, further comprising:
applying a coating to an outer surface of the tubular body to inhibit entry of sound waves into the channel from locations other than the pair of ends.

17. The method of claim 12, wherein the first aperture has a different width than the second aperture.

18. A method for acquiring audio data indicative of sounds internal to a living body, the method comprising:
securing the apparatus of claim 1 to a housing of an electronic device such that the proximal interface is positioned adjacent to a hole through which sound waves travel to reach a microphone; and
locating the electronic device such that the distal interface is positioned adjacent to an anatomical region of the living body.

19. The method of claim 18, wherein said securing comprises:
removing a liner from the apparatus of claim 1 so as to expose an adhesive film along the proximal interface.

20. The method of claim 18, wherein said locating comprises:
holding the electronic device such that the distal interface is directly adjacent to the skin of the living body in the anatomical region.

21. An input unit for an electronic stethoscope system, the input unit comprising:
a printed circuit board having a first side and a second side on which a microphone is mounted;
a structural body that includes
(i) a first section, and
(ii) a second section with an exterior surface that defines a resonator cavity through which sound waves are guided toward the microphone when the structural body is placed against a surface of a living body; and
a tubular body that is comprised of a deformable material and is situated between the second side of the printed circuit board and the second section of the structural body.

22. The input unit of claim 21, wherein the tubular body includes—
a first interface through which the sound waves are collected,
a second interface through which the sound waves are presented to the microphone, and
a channel that is defined by an inner surface and extends between the first and second interfaces, so as to allow the sound waves to travel from the first interface to the second interface.

23. The input unit of claim 21, wherein the deformable material is capable of deforming from an original form under pressure and regaining the original form upon removal of the pressure.

24. The input unit of claim 21, wherein the deformable material is nylon foam.

25. The input unit of claim 21, wherein the tubular body has a height of 2-4 millimeters.

26. The input unit of claim 21, wherein the tubular body has a density of less than 0.4 grams per cubic centimeter.

27. The input unit of claim 21, further comprising:
an adhesive that is situated between the tubular body and the second section of the structural body.

28. The input unit of claim 27, wherein the adhesive is in the form of a double-sided tape comprising a polyethylene terephthalate backing and an acrylic adhesive.

29. The input unit of claim 27, wherein the adhesive is in the form of a gel.

30. The input unit of claim 21, wherein the microphone is configured to product audio data indicative of sounds internal to the living body, and wherein the input unit further comprises:
a transceiver configured to effect a communication channel to facilitate communication with a destination across a network; and
a processor configured to:
forward the audio data to the transceiver for wireless transmission to the destination via the communication channel.

31. The input unit of claim 30, wherein the microphone is configured to product audio data indicative of sounds internal to the living body, and wherein the input unit further comprises:
a second microphone configured to produce second audio data indicative of sounds external to the living body.

32. The input unit of claim 31, wherein the processor is further configured to:
forward the second audio data to the transceiver for wireless transmission to the destination via the communication channel.

33. The input unit of claim 32, wherein the processor is further configured to:
append metadata that identifies the input unit to the audio data and the second audio data.

34. The input unit of claim 21, wherein the resonator cavity has an outer opening across which a diaphragm extends and an inner opening toward which the microphone is oriented.
